(19) United States
(12) Patent Application Publication
(10) Pub. No.: US 2010/0056862 A1
(43) Pub. Date: Mar. 4, 2010

(54) ACCESS NEEDLE FOR NATURAL ORIFICE TRANSLUMENAL ENDOSCOPIC SURGERY

(75) Inventor: Gregory J. Bakos, Mason, OH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/203,458

(22) Filed: Sep. 3, 2008

(51) Int. Cl. A61M 5/32; A61B 1/018

(52) U.S. Cl. 600/106; 604/272; 600/104

(57) ABSTRACT

A translumenal access device may comprise a cannula defining a first lumen and a hollow needle. The hollow needle may be positioned within the cannula. The hollow needle may comprise a first portion including a sharpened rigid distal portion with a first column strength. The hollow needle also may comprise a second portion including a floppy portion with a second column strength. The second portion may be disposed just proximal to the first portion. The first column strength may be greater than the second column strength. The first column strength may be sufficient to penetrate tissue. The second column strength may allow the second portion to buckle to prevent the hollow needle from further penetrating tissue.

### BACKGROUND

The present application relates to endoscopic needles and more particularly to an improved endoscopic needle that helps to prevent accidental injury to nearby anatomical structures during tissue penetration. Such tissue penetration may occur when a surgeon uses the endoscopic needle assembly to gain access to the peritoneal cavity using translumenal access procedures.

Access to the abdominal cavity may be required for diagnostic and therapeutic endeavors for a variety of medical and surgical diseases. Historically, abdominal access has required a formal laparotomy to provide adequate exposure. Such procedures, which require incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a scar if it can be avoided.

Minimally invasive procedures are desirable because such procedures can reduce pain and provide relatively quick recovery times as compared with conventional open medical procedures. Many minimally invasive procedures are performed with an endoscope (including without limitation laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. The trocar must pass through several layers of overlapping tissue/muscle before reaching the abdominal cavity.

Still less invasive treatments include those that are performed through insertion of an endoscope through a natural body orifice to a treatment region. Examples of this approach include, but are not limited to, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient (e.g., mouth, anus, vagina) are known as Natural Orifice Translumenal Endoscopic Surgery (NOTES)™ procedures. Medical instruments such as endoscopic needles may be introduced through the working channel of a flexible endoscope, which typically has a diameter in the range of about 2.5 to about 4 millimeters.

These minimally invasive surgical procedures have changed some of the major open surgical procedures such as gall bladder removal, or a cholecystectomy, to simple outpatient surgery. Consequently, the patient's recovery time has changed from weeks to days. These types of surgeries are often used for repairing defects or for the removal of diseased tissue or organs from areas of the body such as the abdominal cavity.

An issue typically associated with current endoscopic needles is the risk that nearby organs may be accidentally injured by the endoscopic needle. The physician normally cannot see anatomical structures on the distal side of the tissue layers when the endoscopic needle is being pushed through the tissue layers. Therefore, there is a risk that adjacent organs may be accidentally injured by the penetrating needle.

There is a need for an improved endoscopic needle that helps to prevent accidental injury to nearby anatomical structures during tissue penetration.

The foregoing discussion is intended only to illustrate some of the shortcomings present in the art at the time, and should not be taken as a disavowal of claim scope.

### FIGURES

The novel features of the various embodiments are set forth with particularity in the appended claims. The various embodiments, however, both as to organization and methods of operation, may be best understood by reference to the following description, taken in conjunction with the accompanying drawings as follows.

FIG. 1 illustrates a flexible endoscopic portion of one embodiment of a gastroscope inserted into the upper gastrointestinal tract of a patient.

FIG. 2 is partial perspective view of a portion of the endoscope.

FIG. 3A is a side view of one embodiment of an endoscopic needle.

FIG. 3B is an alternate side view of the embodiment of the endoscopic needle of FIG. 3A.

FIG. 3C is a cross-sectional view of one embodiment of a helical slit.

FIG. 3D is a cross sectional view of one embodiment of a helical slit.

FIG. 3E is a cross-sectional view of one embodiment of a helical slit.

FIG. 4 is a side view of one embodiment of an endoscopic needle assembly with the embodiment of the endoscopic needle of FIG. 3A placed within a cannula.

FIG. 5 is a side view of FIG. 4 placed against a portion of tissue.

FIG. 6 is a side view of FIG. 4 with the embodiment of the endoscopic needle of FIG. 3 extended from the cannula to penetrate the portion of tissue.

FIG. 7 is a side view of FIG. 4 with the embodiment of the endoscopic needle of FIG. 3 fully penetrating the portion of tissue.

FIG. 8 is a side view of an alternative embodiment of an endoscopic needle assembly with the embodiment of the endoscopic needle of FIG. 3A placed within a cannula with a guide wire extending into the endoscopic needle.

FIG. 9 is a side view of FIG. 8 with the embodiment of the endoscopic needle of FIG. 3 extended from the cannula to penetrate the portion of tissue.

FIG. 10 is a side view of FIG. 8 with the embodiment of the endoscopic needle of FIG. 3 extended from the cannula to further penetrate the portion of tissue.

FIG. 11 is a side view of FIG. 8 with the embodiment of the endoscopic needle of FIG. 3 extended from the cannula to fully penetrate the portion of tissue.

FIG. 12 is a side view of one embodiment of an endoscopic needle.

FIG. 13 is a side view of one embodiment of an endoscopic needle.

FIG. 14 is a side view of one embodiment of an endoscopic needle.

FIG. 15 is a side view of one embodiment of an endoscopic needle.

FIG. 16 is a side view of one embodiment of an alternative tissue penetrating tip of an endoscopic needle.

FIG. 17 is a perspective view of one embodiment of a surgical instrument that is adapted for use with the embodiment of the endoscopic needle assembly of FIG. 8 to help prevent injury to nearby anatomical structures during endoscopic needle penetration.

### DESCRIPTION

Before explaining the various embodiments in detail, it should be noted that the embodiments are not limited in their application or use to the details of construction and arrangement of parts illustrated in the accompanying drawings and description. The illustrative embodiments may be implemented or incorporated in other embodiments, variations and modifications, and may be practiced or carried out in various ways. For example, the endoscopic needle configurations disclosed below are illustrative only and not meant to limit the scope or application thereof. Furthermore, unless otherwise indicated, the terms and expressions employed herein have been chosen for the purpose of describing the illustrative embodiments for the convenience of the reader and not to limit the scope thereof.

A physician may fully penetrate an endoscopic needle through tissue layers of an organ in order to allow access to the peritoneal cavity of the patient, for example. The physician normally cannot see anatomical structures on the distal side of the tissue layers through the endoscope and therefore may accidentally injure nearby organs with the penetrating needle. An aspect of the endoscopic needle is provided to help prevent such accidental injury.

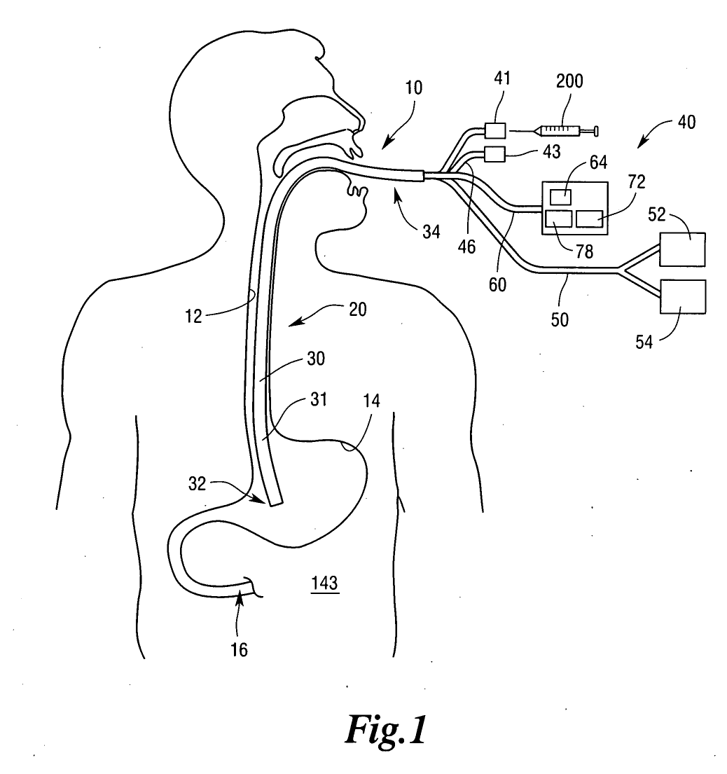

Newer procedures have developed which may even be less invasive than the laparoscopic procedures used in earlier surgical procedures. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as NOTES™. NOTES™ is a surgical technique whereby operations can be performed trans-orally (as depicted in FIG. 1), trans-anally, and/or trans-vaginally.

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of the various embodiments is defined solely by the claims. The features illustrated or described in connection with one embodiment may be combined with the features of other embodiments. Such modifications and variations are intended to be included within the scope of the claims.

It will be appreciated that the terms “proximal” and “distal” are used herein with reference to a clinician gripping the surgical instrument. Thus, the endoscopic needle assemblies are distal with respect to the handle assemblies of the surgical instrument. It will be further appreciated that, for convenience and clarity, spatial terms such as “top” and “bottom” also are used herein with respect to the clinician gripping the handle. However, surgical instruments are used in many orientations and positions, and these terms are not intended to be limiting and absolute.

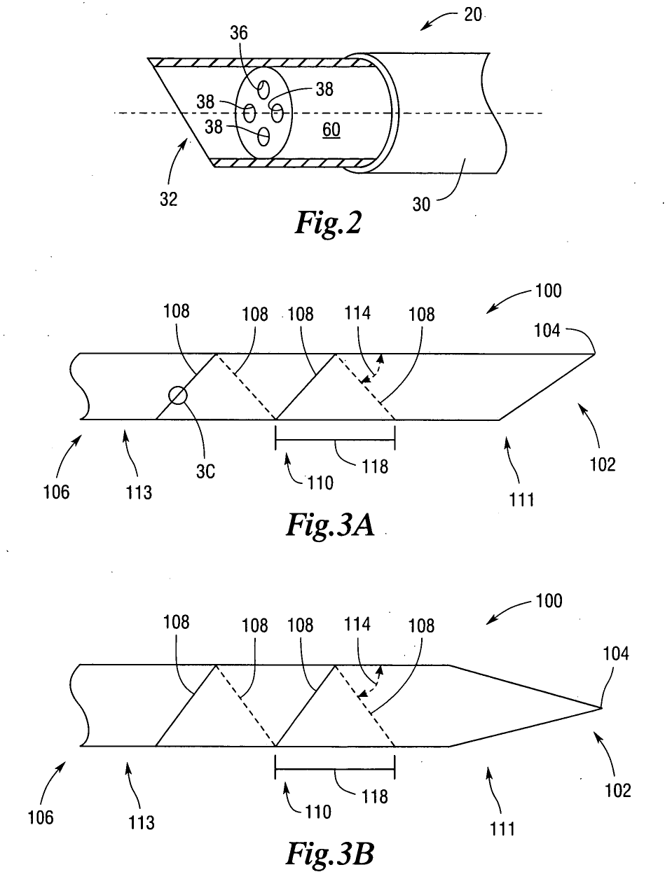

FIG. 1 illustrates a flexible endoscopic portion 31 of a gastroscope inserted into the upper gastrointestinal tract of a patient. FIG. 2 is a drawing of the distal portion 32 of an endoscope. FIG. 1 illustrates, in general form, one embodiment of a surgical instrument 20 that can be inserted through a natural orifice such as the mouth 10 and esophagus 12 into the stomach 14 to establish a surgical opening in the stomach 14 for performing a surgical operation such as a gall bladder removal, or a cholecystectomy. As shown in FIG. 2, the surgical instrument 20 may comprise a hollow outer sleeve 30 that has a distal end 32 and a proximal end 40 (FIG. 1). In various embodiments, the hollow outer sleeve 30 may be fabricated from, for example, nylon or high density polyethylene plastic. In various embodiments, the hollow outer sleeve 30 can serve to define various tool-receiving passages 38 that extend from the natural orifice 10 to the surgical site. In addition, the hollow outer sleeve may serve to define a viewing port 36. An endoscope 60 (FIG. 1) may be used for viewing a surgical site within the patient's body. Various cameras and/or lighting apparatuses may be inserted into the viewing port 36 of the endoscope 60 to provide the surgeon with a view of the surgical site.

As shown in FIG. 1, in various embodiments, one of the tools or surgical instruments that can be accommodated in the tool-receiving passage 38 is a hollow vacuum/air tube 50 that may communicate with at least one of a vacuum source 52 and a source of pressurized air 54. In one embodiment, the vacuum/air tube 50 can be sized to receive therein another surgical instrument in the form of the endoscope 60. A variety of different types of endoscopes are known and, therefore, their specific construction and operation will not be discussed in great detail herein. In various embodiments, the endoscope 60 may operably support a video camera that communicates with a video display unit 64 that can be viewed by the surgeon during the operation. In addition, the endoscope 60 may further have a fluid-supply lumen therethrough that is coupled to a source 72 of water, saline solution, and/or any other suitable fluid and/or an air supply lumen that is coupled to the source of air 78.

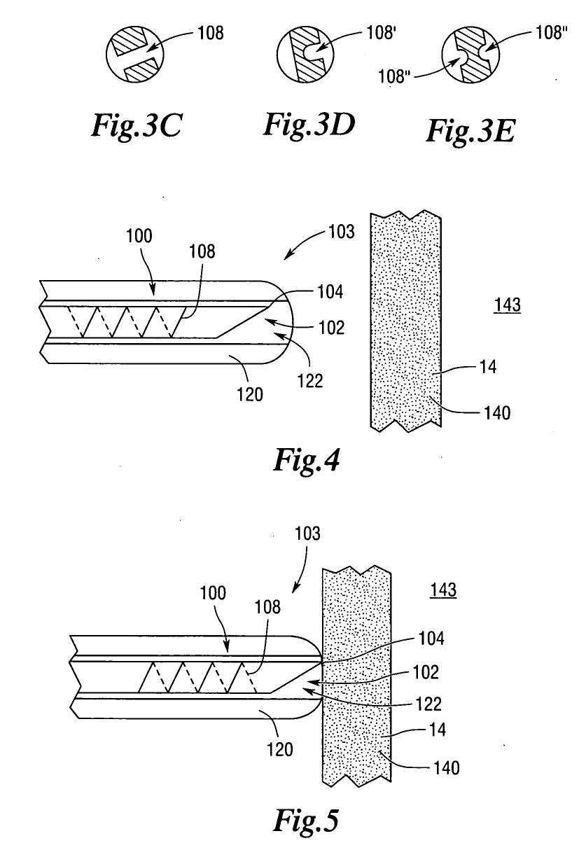

FIG. 3A is a side view of one embodiment of an endoscopic needle 100. FIG. 3B is an alternate side view of one embodiment of the endoscopic needle 100. In various embodiments, the endoscopic needle 100 may be formed of a tube which may have a channel extending from a proximal end 106 of the endoscopic needle 100 to a distal end 102 of the endoscopic needle 100. In one embodiment, the endoscopic needle 100 may be hollow. The distal end 102 of the endoscopic needle 100 may comprise a tissue penetrating tip 104. As shown in FIG. 3A-3B, the tissue-penetrating tip 104 may be located at the distal end 102 at the outside of the diameter of the endoscopic needle 100. The tissue penetrating tip 104 may be cut and/or ground so that the sharp portion of the tissue penetrating tip is located at the outer edge of the diameter of the endoscopic needle. The endoscopic needle 100 may be ground to form the tissue penetrating tip 104. The endoscopic needle 100 may be fabricated from medical grade stainless steel, nitinol, or polyetheretherketon (PEEK) hypodermic tubing or any other suitable medical grade material which may include metal and/or plastic suitable for medical applications, for example. Alternatively, the endoscopic needle 100 may be formed from an alternate type of metallic or polymeric tube and attached to a cannulated needle, or tube, (not shown), such as by bolting, screwing, welding, crimping, gluing or any other suitable method. The endoscopic needle 100 may have an outer diameter in the range of about 0.010 inches to about 0.050 inches. For example, the endoscopic needle 100 may be formed from medical grade stainless steel hypodermic tubing having an outer diameter of approximately 0.035 inches. The endoscopic needle 100 may have an inner diameter in the range of about 0.005 inches to about 0.045 inches. For example, the endoscopic needle 100 may have an inner diameter of 0.020 inches.

As shown in FIGS. 3A-3B, the endoscopic needle 100 may comprise a first portion 111 at or near the distal end 102, a second portion 110 proximal to the first portion 111, and a third portion 113 proximal to the second portion 110 at or near the proximal end. The first portion 111 may have column strength sufficient to allow the tissue penetrating tip 104 of the endoscopic needle 100 to penetrate tissue. The second portion 110 may be fabricated such that the second portion 110 may have column strength that is less than the column strength of the first portion 111. The column strength of the second portion 110 may be weakened to a degree that the second portion 110 may exhibit flexible and/or floppy properties.

FIG. 3C is a cross-sectional view of one embodiment of a helical slit 108. FIG. 3D is a cross-sectional view of one embodiment of a helical slit 108′. FIG. 3E is a cross-sectional view of one embodiment of a helical slit 108″. In various embodiments, the second portion 110 of the endoscopic needle 100 may comprise the helical slit 108. The helical slit 108 may extend from the distal end of the second portion 110 to the proximal end of the second portion 110. The helical slit 108 may extend about the periphery of the tubular material of the endoscopic needle 100. As shown in FIG. 3C, in one embodiment, the helical slit 108 may completely penetrate the tube of the endoscopic needle 100 from the outer diameter to the inner diameter. As illustrated in FIG. 3D, in one embodiment, the helical slit 108′ may be formed by scoring the outer surface of the tube without completely penetrating the wall of the tube. As shown in FIG. 3E, in one embodiment, the helical slit 108″ may be formed by scoring both the inner surface of the tube and the outer surface of the tube without completely penetrating the wall of the tube. The helical slit 108 may be cut into the endoscopic needle at a specified angle 114. The specified angle 114 may be in the range of about 10 degrees to about 80 degrees. The specified angle 114 may vary depending upon the degree of flexibility, or floppiness, required of the second portion 110. The helical slit 108 may have a length 118. The length 118 of the helical slit 108 may be in the range of about 0.19 inches to about 0.79 inches (or about 5 mm to about 20 mm). The length 118 of the helical slit 108 may vary depending upon the degree of flexibility, or floppiness, required of the second portion 110. The specified angle 114 and/or the length 118 of the helical slit 108 may vary along the length of the second portion 110.

In one embodiment, the third portion 113 may have column strength that may allow the endoscopic needle 100 to flexibly extend along the length of the endoscope 60. The third portion 113 may extend from the proximal end of the second portion 110 to the proximal portion of the third portion 113. In one embodiment, the proximal end of the third portion 113 may extend to the handle portion of the endoscope 60. In an alternative embodiment, the third portion 113 may only extend to a tube (not shown) which may extend to the handle portion of the endoscope 60. The third portion 113 may be attached to the tube through gluing, welding, bolting, screwing, or any other suitable attachment means.

FIG. 4 is a side view of one embodiment of an endoscopic needle assembly 103 with the endoscopic needle 100 placed within a cannula 120. FIG. 5 is a side view of the endoscopic needle assembly 103 placed against a portion of tissue 140. For example, the tissue 140 may be part of the stomach 14 wall (FIG. 1). In various embodiments, the cannula 120 may be used to support the endoscopic needle 100 as the endoscopic needle penetrates a portion of the tissue 140. The cannula 120 and the endoscopic needle 100 may be part of a surgical instrument used for translumenal access. The translumenal access device may be configured to be disposed within the working channel 38 (FIG. 2) of the endoscope 60 (FIGS. 1-2). The cannula 120, or catheter, may comprise a central lumen 122 and a secondary lumen (not shown). The cannula 120 may be fabricated from nylon, polyvinylchloride (PVC), urethane, or any other suitable polymer. The endoscopic needle 100 may be slidably disposed within the central lumen 122 of the cannula 120. The secondary lumen may be in fluid communication with an inflatable member. The secondary lumen may be configured to provide fluid to the inflatable member located on, or near, the cannula 120. As shown in FIG. 5, the cannula 120 may be placed against the portion of the tissue 140 to be punctured by the endoscopic needle 100.

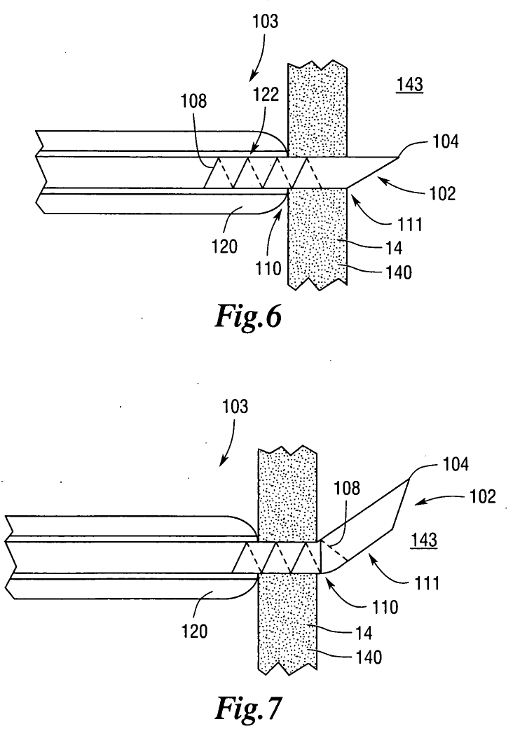

FIG. 6 is a side view of the endoscopic needle assembly 103 with the first portion 111 of the endoscopic needle 100 extended distally from the cannula 120 to penetrate the portion of tissue 140. In various embodiments, the endoscopic needle 100 may be forced to move distally from the cannula 120. The movement of the endoscopic needle 100 may be controlled by the operator of the surgical instrument. As the operator advances the endoscopic needle 100 distally, the first portion 111 of the endoscopic needle 100 may penetrate the tissue 140 of the stomach wall 14 and enter the peritoneal cavity 143. As shown in FIG. 6, the second portion 110, or the flexible and/or floppy portion, of the endoscopic needle 100 may still be retained within the central lumen 122 of the cannula 120.

FIG. 7 is a side view of the endoscopic needle assembly 103 with the first portion 111 of the endoscopic needle 100 fully penetrating the portion of tissue 140. Once the operator advances the first portion 111 of the endoscopic needle 100 to fully penetrate the tissue 140, the operator may continue to advance the endoscopic needle 100. As previously discussed, with reference to conventional endoscopic needles, advancing the endoscopic needle past the point of puncture may cause unintended injury to adjacent organs, blood vessels, or any other tissue within a patient's body. In various embodiments, as the operator forces the endoscopic needle 100 past the point of puncture, the second portion 110 of the endoscopic needle 100 may advance from the cannula 120. As shown in FIG. 7, the degree of flexibility/floppiness of the second portion 110 may cause the endoscopic needle 100 to bend or be diverted from adjacent organs, blood vessels and/or any other tissue within a patient's body. This may occur due to a lack of column strength in the second portion 110 of the endoscopic needle 100. The column strength of the second portion 110 may be insufficient to puncture additional tissue. The column strength of the second portion 110 may be such that the endoscopic needle 100 buckles, or bends, warps, or is caused to give way, for example, with substantially no force, or little force, applied to the distal end 402 of the endoscopic needle 100. In one embodiment, the column strength of the second portion 110 may be selected such that the endoscopic needle 100 bends when subjected to a force that is equal to or greater than a force required to puncture the portion of tissue 140.

In various embodiments, once the endoscopic needle 100 has penetrated the tissue 140, a surgical instrument may be inserted through the penetration point in the tissue 140 until the inflatable member (not shown) extends from one side of the penetration to another side of the penetration. When the inflatable member is in position, the inflatable member may be inflated by the surgeon to expand the opening. Once the inflatable member has been inflated, the distal end 32 (FIG. 1) of the endoscope 60 (FIG. 1) may be placed at a proximal end of the inflatable member. Then, the inflatable member and the distal end 32 of the endoscope 60 may be forced through the opening. The inflatable member may then be deflated. At this point, the surgical instrument may be removed from the working channel 38 (FIG. 2) of the endoscope 60.

The configuration of the endoscopic needle 100 may vary depending upon the particular application (i.e., the tissue to be penetrated). The first portion 111, the second portion 110, and the third portion 113 may be adjusted according to the depth of penetration required. For example, if the endoscopic needle 100 is required to puncture 5 mm, the second portion 110 of the endoscopic needle 100 may comprise a helical slit pattern that increases in frequency at a location 5 mm from the distal end of the first portion 111 to make the second portion 110 especially floppy at that point. The endoscopic needle 100 may be configured to penetrate tissue ranging from a depth of approximately 0.02 inches (or approximately 0.5 mm) to approximately 0.5 inches (or approximately 13 mm).

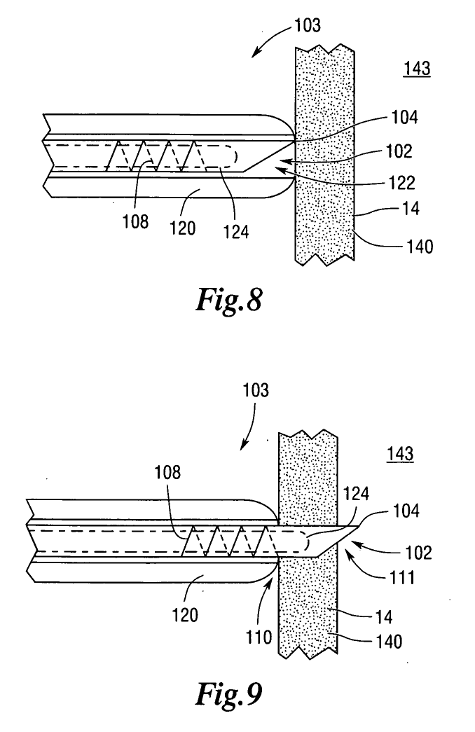

FIG. 8 is a side view of an alternative embodiment of an endoscopic needle assembly 103 with the endoscopic needle 100 placed within the cannula 120 with a guide wire 124 extending into the endoscopic needle 100. FIG. 9 is a side view of the endoscopic needle assembly 103 with the first portion 111 of the endoscopic needle 100 extended from the cannula 120 to penetrate the portion of tissue 140. In various embodiments of the endoscopic needle assembly 103, the endoscopic needle assembly 103 also may comprise the guide wire 124. The guide wire 124 may be configured to be slidably retained within the channel in the endoscopic needle 100. The guide wire 124 may be fabricated from nytenol, or any other suitable material, with a TEFLON®, or any other suitable coating, placed upon the guide wire 124. In various embodiments, the guide wire 124 may be formed with a blunt tip at the distal end of the guide wire 124 to prevent the guide wire 124 from puncturing the tissue 140. The guide wire 124 may be flexible enough to travel along the length of the flexible endoscope 60 (FIG. 1) but may have column strength greater than the column strength of the second portion 110 of the endoscopic needle 100. The operator may control the guide wire 124 from the proximal end of the endoscope 60. The operator may have the ability to extend the guide wire 124, or to move the guide wire 124 distally. In addition, the operator may have the ability to retract the guide wire 124, or move the guide wire 124 proximally. The guide wire 124 may be advanced by the operator until the guide wire 124 is at or near the distal end 102 of the endoscopic needle 100 prior to tissue 140 penetration. When the cannula 120 is in place at or near the tissue 140 to be penetrated, the endoscopic needle 100 and the guide wire 124 may be advanced by the operator. The endoscopic needle 100 and the guide wire 124 may be advanced until the first portion 111 of the endoscopic needle 100 punctures the tissue 140.

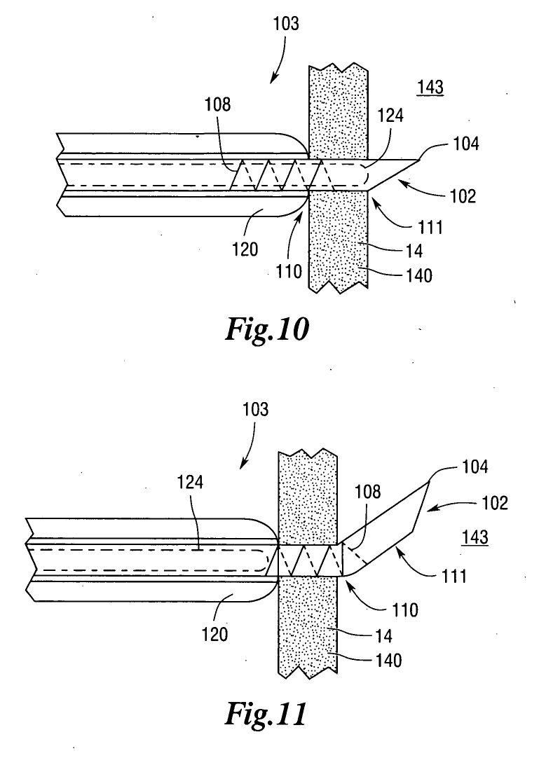

FIG. 10 is a side view of the endoscopic needle assembly 103 with the first portion 111 of the endoscopic needle 100 and the guide wire 124 extended distally from the cannula 120 to fully penetrate the portion of tissue 140. FIG. 11 is a side view of the endoscopic needle assembly 103 with the endoscopic needle 100 extended distally from the cannula 120 fully penetrating the portion of tissue 140 and the guide wire 124 retracted into the endoscopic needle 100. As shown in FIG. 10, the first portion 111 of the endoscopic needle 100 and the guide wire 124 may be advanced until the endoscopic needle 100 punctures the tissue 140. The guide wire 124 may provide support to the first portion 111 of the endoscopic needle 100 such that the first portion 111 of the endoscopic needle 100 has sufficient column strength to penetrate the tissue 140. In various embodiments, the guide wire 124 also may comprise a flexible/floppy portion that when combined with or aligned with the first portion 111 could cause a change in the column strength of the first portion 111. As shown in FIG. 11, once the first portion 111 of the endoscopic needle 100 and the guide wire 124 have penetrated the tissue 140, the guide wire 124 may be retracted into the endoscopic needle 100.

In one embodiment, the guide wire 124 may be retracted to a position proximal to the second portion 110 of the endoscopic needle 100. With the guide wire 124 retracted proximally to a position proximal to the second portion 110, the second portion 110 may not have enough column strength to penetrate additional tissue. In various embodiments, as the operator forces the endoscopic needle 100 past the point of puncture, the second portion 110 of the endoscopic needle 100 may advance from the cannula 120. As previously discussed, and shown in FIG. 11, the degree of flexibility/floppiness of the second portion 110 may cause the endoscopic needle 100 to bend or be diverted from adjacent organs, blood vessels, and/or other tissue of the patient. This may occur due to a lack of column strength in the second portion 110 of the endoscopic needle 100. As previously discussed, the column strength of the second portion 110 may be insufficient to puncture additional tissue. The column strength of the second portion 110 may be such that the endoscopic needle 100 buckles, or bends, warps, or is caused to give way, for example, with substantially no force, or little force, applied to the distal end 102 of the endoscopic needle 100. In one embodiment, the column strength of the second portion 110 may be selected such that the endoscopic needle 100 bends when subjected to a force that is equal to or greater than a force required to puncture the portion of tissue 140.

A person skilled in the art will recognize that the various embodiments described hereinafter may be used in conjunction with the surgical instrument embodied in FIGS. 4-11.

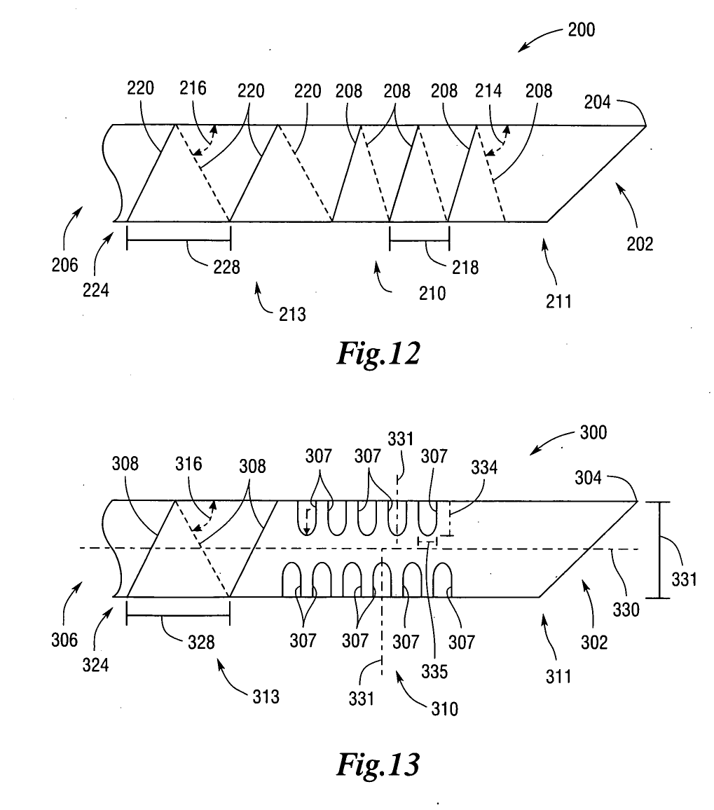

FIG. 12 is a side view of one embodiment of an endoscopic needle 200. In various embodiments, the endoscopic needle 200 may be formed of a tube comprising a channel extending from a proximal end 206 of the endoscopic needle 200 to a distal end 202 of the endoscopic needle 200. The endoscopic needle 200 may be hollow. The distal end 202 of the endoscopic needle 200 may comprise a tissue penetrating tip 204. The endoscopic needle 200 may be ground to form the tissue penetrating tip 204. The endoscopic needle 200 may be fabricated from medical grade stainless steel, nitinol, or PEEK hypodermic tubing or any other suitable material which may include medical grade metal and/or plastic for use in medical applications. Alternatively, the endoscopic needle 200 may be formed from an alternate type of metallic or polymeric tube and attached to a cannulated needle, or tube, (not shown), by bolting, screwing, welding, crimping, gluing or any other suitable method. The endoscopic needle 200 may have an outer diameter in the range of about 0.010 inches to about 0.050 inches and an inner diameter in the range of about 0.005 inches to about 0.045 inches.

As shown in FIG. 12, the endoscopic needle 200 may comprise a first portion 211 at or near the distal end 202, a second portion 210 proximal to the first portion 211, a third portion 213 proximal to the second portion 210, and a fourth portion 220 proximal to the third portion 213 at or near the proximal end 206. The first portion 211 may have column strength sufficient to allow the tissue penetrating tip 204 of the endoscopic needle 200 to penetrate tissue. The second portion 210 may be fabricated such that the second portion 210 may have column strength that is less than the column strength of the first portion 211. The column strength of the second portion 210 may be weakened to a degree that the second portion 210 may exhibit flexible and/or floppy properties.

In various embodiments, the second portion 210 of the endoscopic needle 202 may comprise a first helical slit 208. The first helical slit 208 may extend from the distal end of the second portion 210 to the proximal end of the second portion 210. The first helical slit 208 may extend about the periphery of the tubular material of the endoscopic needle 202. The first helical slit 208 may completely penetrate the tube of the endoscopic needle 200 from the outer diameter to the inner diameter or may simply score the outer surface and/or the inner surface of the tube without completely penetrating the wall of the tube. The first helical slit 208 may be cut into the endoscopic needle 202 at a first specified angle 214. The first specified angle 214 may be in the range of about 10 degrees to about 80 degrees. The first specified angle 214 may vary depending upon the degree of flexibility, or floppiness, required of the second portion 210. The first helical slit 208 also may have a first length 218. The first length 218 of the first helical slit 208 may be in the range of about 0.19 inches to about 0.79 inches (or about 5 mm to about 20 mm). The first length 218 of the first helical slit 208 may vary depending upon the degree of flexibility, or floppiness, required of the second portion 210. The first specified angle 214 and/or the first length 218 of the first helical slit 208 may vary along the length of the second portion 210.

In one embodiment, the third portion 213 of the endoscopic needle 202 may comprise a second helical slit 220. The second helical slit 220 may extend from the distal end of the third portion 213 to the proximal end of the third portion 213. The second helical slit 220 may extend about the periphery of the tubular material of the endoscopic needle 202 in a manner similar to the first helical slit 208. The second helical slit 220 may completely penetrate the tube of the endoscopic needle 200 from the outer diameter to the inner diameter or may simply score the outer surface and/or the inner surface of the tube without completely penetrating the wall of the tube. The second helical slit 220 may be cut into the endoscopic needle 202 at a second specified angle 216. The second specified angle 216 may be in the range of about 10 degrees to about 80 degrees. The second specified angle 216 may vary depending upon the degree of flexibility, or floppiness, required of the third portion 213. The second helical slit 220 may have a second length 228. The second length 228 of the second helical slit 220 may be in the range of about 0.19 inches to about 0.79 inches (or about 5 mm to about 20 mm). The second length 228 of the second helical slit 220 may vary depending upon the degree of flexibility, or floppiness, required of the second portion 213. For example, the endoscopic needle 200 may require that the second portion 210 is more flexible (i.e., floppy or limp) than the third portion 213. The second specified angle 216 and/or the second length 228 of the second helical slit 220 may vary along the length of the third portion 213. In various embodiments, the second helical slit 220 may simply be an extension of the first helical slit 208. In various other embodiments, the second helical slit 220 may be completely separate from the first helical slit 208.

In one embodiment, the fourth portion 224 may have column strength that may allow the endoscopic needle 202 to flexibly extend along the length of the endoscope 60 (FIG. 1). The fourth portion 224 may extend from the proximal end of the third portion 213 to the proximal portion of the fourth portion 224. In one embodiment, the proximal end of the fourth portion 224 may extend to the handle portion of the endoscope 60. In an alternative embodiment, the fourth portion 224 may extend only to a tube (not shown), which may extend to the handle portion of the endoscope 60. The fourth portion 224 may be attached to the tube through gluing, welding, bolting, screwing, or any other suitable attachment means.

FIG. 13 is a side view of one embodiment of an endoscopic needle 300. In various embodiments, the endoscopic needle 300 may be formed of a tube which may have a channel extending from a proximal end 306 of the endoscopic needle 300 to a distal end 302 of the endoscopic needle 300. In one embodiment, the endoscopic needle 300 may be hollow. In various other embodiments (not shown), the endoscopic needle 300 may be formed from a solid cylinder of any suitable cross-section including a circular cylinder or an elliptic cylinder. The distal end 302 of the endoscopic needle 300 may comprise a tissue penetrating tip 304. The endoscopic needle 300 may be ground to form the tissue penetrating tip 304. The endoscopic needle 300 may be fabricated from medical grade stainless steel, nitinol, or PEEK hypodermic tubing or any other suitable material which may include medical grade metal and/or plastic for medical applications. Alternatively, the endoscopic needle 300 may be formed from an alternate type of metallic or polymeric tube and attached to a cannulated needle, or tube, (not shown), by bolting, screwing, welding, crimping, gluing or any other suitable method. The endoscopic needle 300 may have an outer diameter in the range of about 0.010 inches to about 0.050 inches and an inner diameter in the range of about 0.005 inches to about 0.045 inches.

As shown in FIG. 13, the endoscopic needle 300 may comprise a first portion 311 at or near the distal end 302, a second portion 310 proximal to the first portion 311, a third portion 313 proximal to the second portion 310, and a fourth portion 320 proximal to the third portion 313 at or near the proximal end 306. The first portion 311 may have column strength sufficient to allow the tissue penetrating tip 304 of the endoscopic needle 300 to penetrate tissue. The second portion 310 may be fabricated such that the second portion 310 may have column strength that is less than the column strength of the first portion 311. The column strength of the second portion 310 may be weakened to a degree that the second portion 310 may exhibit flexible and/or floppy properties.

In various embodiments, the second portion 310 of the endoscopic needle 302 may comprise a plurality of slits 307 formed in the second portion 310 such as a notch, an indentation, or any other suitable configuration. The slits 307 of the second portion 310 may be made to remove material from the second portion 310. As shown in FIG. 13, the slits 307 may be formed such that they have a radius r and an axis 331. In other embodiments (not shown), the slits 307 may be formed in a v-shaped notch or any other suitable shape. The slits 307 may be disposed on opposite sides of an axis 330. In one embodiment, the axes 331 of the slits 307 may be offset (as shown in FIG. 13). In an alternative embodiment, the axis 331 of the slits 307 may be substantially aligned (not shown). The slits 307 may extend inwards from a periphery 331 of the second portion 310 towards the axis 330. The slits 307 may extend into the second portion 310 for a depth 334 less than a diameter 332 of the second portion 310. For example, in one embodiment, the depth 334 may be approximately ¼ of the diameter 332. In an alternative embodiment, the depth 334 may be approximately ⅓ of the diameter 332. The depth 334 of the slits 307 may vary along the length of the axis 330. In addition, the slits 307 may have a width 335 associated with each slit 307.

The slits 307 may extend along the length of the second portion 310 from the distal end of the second portion 310 to the proximal end of the second portion 310. The slits 307 may be made in the endoscopic needle 302 such that the axis 331 are substantially perpendicular to the axis 330. In an alternative embodiment, the slits 307 may be made such that the axes 331 meet the axis 332 at a first specified angle (not shown). The depth 334, the width 335, and/or the radius r of the slits 307 may vary depending upon the degree of flexibility, or floppiness, required of the second portion 310. The depth 334, the width 335, and/or the radius r of the slits 307 may vary along the length of the second portion 310.

In one embodiment, the third portion 313 of the endoscopic needle 302 may comprise a first helical slit 308. The first helical slit 308 may extend from the distal end of the third portion 313 to the proximal end of the third portion 313. The first helical slit 308 may extend about the periphery of the tubular material of the endoscopic needle 302. The first helical slit 308 may completely penetrate the tube of the endoscopic needle 300 from the outer diameter to the inner diameter or may simply score the outer surface and/or the inner surface of the tube without completely penetrating the wall of the tube. The first helical slit 308 may be cut into the endoscopic needle 302 at a first specified angle 316. The first specified angle 316 may be in the range of about 10 degrees to about 80 degrees. The first specified angle 316 may vary depending upon the degree of flexibility, or floppiness, required of the third portion 313. The first helical slit 308 may have a first length 328. The first length 328 of the first helical slit 308 may be in the range of about 0.19 inches to about 0.79 inches (or about 5 mm to about 20 mm). The first length 328 of the first helical slit 308 may vary depending upon the degree of flexibility, or floppiness, required of the third portion 313. For example, the endoscopic needle 300 may require that the second portion 310 is more flexible (i.e., floppy or limp) than the third portion 313. The first specified angle 316 and/or the first length 328 of the first helical slit 308 may vary along the length of the third portion 313.

In one embodiment, the fourth portion 324 may have column strength that may allow the endoscopic needle 302 to flexibly extend along the length of the endoscope 60 (FIG. 1). The fourth portion 324 may extend from the proximal end of the third portion 313 to the proximal portion of the fourth portion 324. In one embodiment, the proximal end of the fourth portion 324 may extend to the handle portion of the endoscope 60. In an alternative embodiment, the fourth portion 324 may extend only to a tube (not shown) which may extend to the handle portion of the endoscope 60. The fourth portion 324 may be attached to the tube through gluing, welding, bolting, screwing, or any other suitable attachment means.

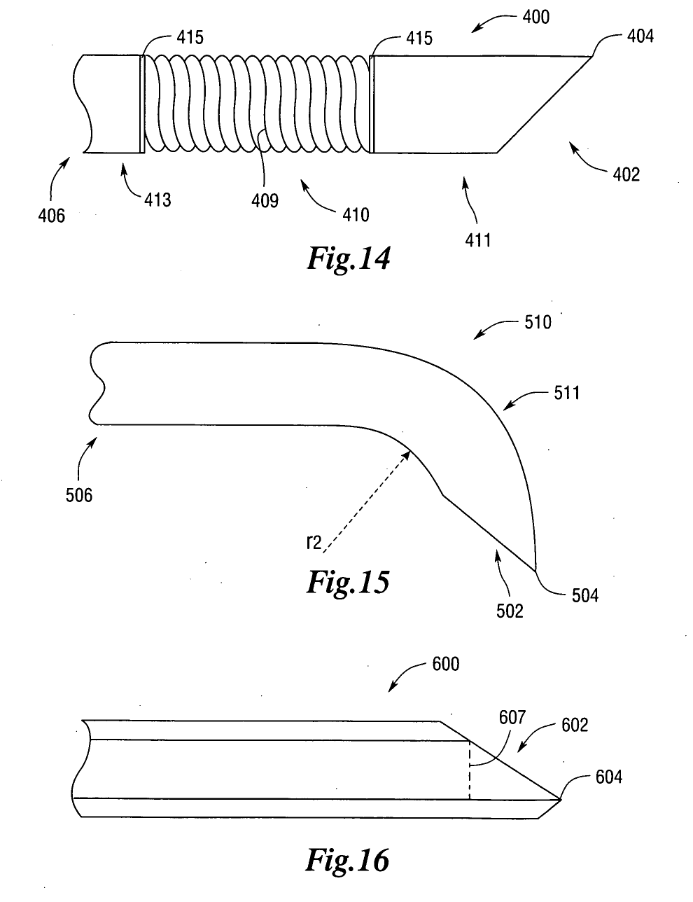

FIG. 14 is a side view of a fourth embodiment of an endoscopic needle 400. In various embodiments, the endoscopic needle 400 may be formed, at least in part, of a tube which may have a channel extending from a proximal end 406 of the endoscopic needle 400 to a distal end 402 of the endoscopic needle 400. The endoscopic needle 400 may be hollow. The distal end 402 of the endoscopic needle 400 may comprise a tissue penetrating tip 404. The endoscopic needle 400 may be ground to form the tissue penetrating tip 404. The endoscopic needle 400 may be fabricated from medical grade stainless steel, nitinol, or PEEK hypodermic tubing or any other suitable material which may include medical grade metal and/or plastic for medical applications. Alternatively, the endoscopic needle 400 may be formed from an alternate type of metallic or polymeric tube and attached to a cannulated needle, or tube, (not shown), by bolting, screwing, welding, crimping, gluing or any other suitable method. The endoscopic needle 400 may have an outer diameter in the range of about 0.010 inches to about 0.050 inches and an inner diameter in the range of about 0.005 inches to about 0.045 inches.

As shown in FIG. 14, the endoscopic needle 400 may comprise a first portion 411 at or near the distal end 402, a second portion 410 proximal to the first portion 411, and a third portion 413 proximal to the second portion 410 at or near the proximal end. The first portion 411 may have column strength sufficient to allow the tissue penetrating tip 404 of the endoscopic needle 400 to penetrate tissue. The second portion 410 may comprise a spring 409. The spring 409 may be a helical spring. The spring 409 may be fabricated such that the second portion 410 may have column strength that is less than the column strength of the first portion 411. The column strength of the second portion 410 may be weakened to a degree that the second portion 410 may exhibit flexible and/or floppy properties. The spring 409 of the second portion 410 may be disposed between the distal end 402 and the proximal end 406 of the endoscopic needle 400. The spring 409 may be attached to the distal end 402 and/or the proximal end 406 through gluing, welding, bolting, screwing, or any other suitable attachment means. In an alternative embodiment, the spring 409 may be integrally formed with the distal end 402 and the proximal end 406.

In one embodiment, the third portion 413 may have column strength that may allow the endoscopic needle 402 to flexibly extend along the length of the endoscope 60 (FIG. 1). The third portion 413 may extend from the proximal end 406 of the second portion 410 to the proximal portion of the third portion 413. In one embodiment, the proximal end 406 of the third portion 413 may extend to the handle portion of the endoscope 60. In an alternative embodiment, the third portion 413 may extend only to a tube (not shown), which may extend to the handle portion of the endoscope 60. The third portion 413 may be attached to the tube through gluing, welding, bolting, screwing, or any other suitable attachment means.

FIG. 15 is a side view of a fifth embodiment of an endoscopic needle 500. In various embodiments, the endoscopic needle 500 may be formed of a tube which may have a channel extending from a proximal end 506 of the endoscopic needle 500 to a distal end 502 of the endoscopic needle 500. The endoscopic needle 500 may be hollow. The distal end 502 of the endoscopic needle 500 may comprise a tissue penetrating tip 504. The endoscopic needle 500 may be ground to form the tissue penetrating tip 504. The endoscopic needle 500 may be fabricated from medical grade stainless steel, nitinol, or PEEK hypodermic tubing or any other suitable material which may include medical grade metal and/or plastic for medical applications. Alternatively, the endoscopic needle 500 may be formed from an alternate type of metallic or polymeric tube and attached to a cannulated needle, or tube, (not shown), by bolting, screwing, welding, crimping, gluing or any other suitable method. The endoscopic needle 500 may have an outer diameter in the range of about 0.010 inches to about 0.050 inches and an inner diameter in the range of about 0.005 inches to about 0.045 inches.

As shown in FIG. 15, the endoscopic needle 500 may comprise a first portion 511 at or near the distal end 502, a second portion 510 proximal to the first portion 511, and a third portion 513 proximal to the second portion 510 at or near the proximal end 506. The first portion 511 may have column strength sufficient to allow the tissue penetrating tip 504 of the endoscopic needle 500 to penetrate tissue. The second portion 510 may be pre-curved with a radius r2. The endoscopic needle 500 may be formed of a suitable superelastic material which is configurable with a preformed curve. The length of the radius r2 may vary according to the required compression strength of the second portion 510. For example, the length of the radius r2 may be about 0.787 inches or about 2 cm. The second portion 510 may have column strength that is less than the column strength of the first portion 511. The column strength of the second portion 510 may be weakened to a degree that the second portion 510 may exhibit flexible and/or floppy properties. The second portion 510 may be disposed between the distal end 502 and the proximal end 506 of the endoscopic needle 500. The second portion 510 may be attached to the distal end 502 and/or the proximal end 506 through gluing, welding, bolting, screwing, or any other suitable attachment means. In an alternative embodiment, the second portion 510 may be integrally formed with the distal end 502 and the proximal end 506.

In one embodiment, the third portion 513 may have column strength that may allow the endoscopic needle 500 to flexibly extend along the length of the endoscope 60. The third portion 513 may extend from the proximal end of the second portion 510 to the proximal portion of the third portion 513. In one embodiment, the proximal end 506 of the third portion 513 may extend to the handle portion of the endoscope 60. In an alternative embodiment, the third portion 513 may extend only to a tube (not shown) which may extend to the handle portion of the endoscope 60. The third portion 513 may be attached to the tube through gluing, welding, bolting, screwing, or any other suitable attachment means.

FIG. 16 is a side view of one embodiment of an alternative tissue penetrating tip 604 of an endoscopic needle 600. In various embodiments, the distal end 602 of the endoscopic needle 600 may comprise a tissue-penetrating tip 604. As shown in FIG. 16, the tissue-penetrating tip 604 may be located at the distal end 602 at the outside of the diameter of the endoscopic needle 600. The tissue-penetrating tip 604 may be cut and/or ground so that the sharp portion of the tissue-penetrating tip 604 is located at the inner edge of the inner diameter 607 of the tubular material of the endoscopic needle 600. The endoscopic needle 600 may be ground to form the tissue penetrating tip 604. The endoscopic needle 600 may be fabricated from medical grade stainless steel, nitinol, or PEEK hypodermic tubing or any other suitable medical grade material, which may include metal and/or plastic suitable for medical, applications, for example. Alternatively, the endoscopic needle 600 may be formed from an alternate type of metallic or polymeric tube and attached to a cannulated needle, or tube, (not shown), by bolting, screwing, welding, crimping, gluing or any other suitable method.

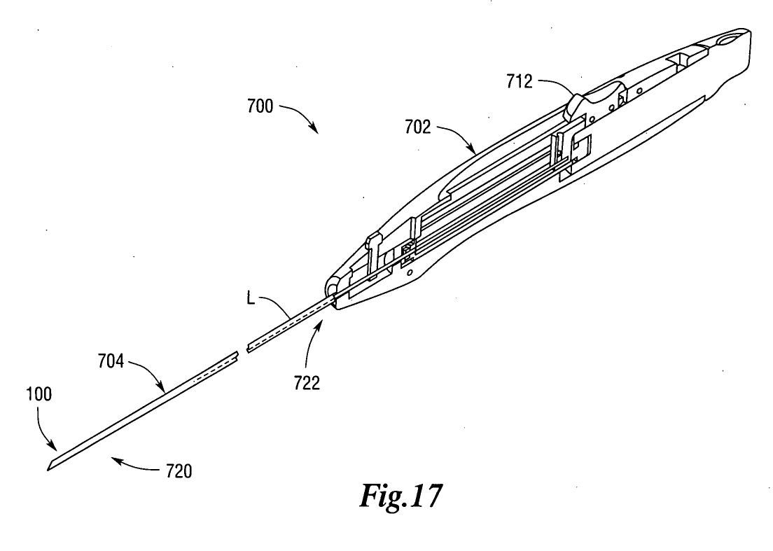

FIG. 17 is a perspective view of an embodiment of a surgical instrument 700 that is adapted for use with the endoscopic needle assembly 103 to help prevent injury to nearby anatomical structures during endoscopic needle 100 penetration. The surgical instrument 700 may include an elongate shaft 704 attached to a handle 702. The shaft 704 may have a distal end 720 and a proximal end 722 defining a longitudinal axis L therebetween. The shaft 704 may be flexible and may be sized for insertion into the working channel of the flexible endoscope 60 (FIG. 1). The surgical instrument 700 may be used in conjunction with any suitable endoscopic needle assembly, such as those previously discussed. The endoscopic needle assembly 103 may be located at the distal end 720 of the shaft 704. The endoscopic needle assembly 103 may be attached to the distal end 720 through any attachment means, which may include bolting, screwing, welding, gluing, fusing, or any other suitable method. The surgical instrument 700 is described next as it may be adapted for use with the endoscopic needle assembly 103, although the surgical instrument 700 may be adapted for use with various suitable endoscopic needle assemblies. The handle 702 may include an actuator 712. A physician may operate the actuator 712 to advance the endoscopic needle 100 and/or the guide wire 124 (FIG. 8) to penetrate into the tissue 140 (FIG. 8). In various embodiments (not shown), the endoscopic needle 100 and the guide wire 124 may be actuated with separate actuators.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam.

Although various embodiments have been described herein, many modifications and variations to those embodiments may be implemented. For example, different types of endoscopic needle assemblies may be employed. In addition, combinations of the described embodiments may be used. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A translumenal access device comprising:
a cannula defining a first lumen; and
a hollow needle positioned within the cannula, the hollow needle comprising a first portion comprising a sharpened rigid distal portion with a first column strength and a second portion comprising a floppy portion with a second column strength, the second portion disposed proximal to the first portion, the first column strength is greater than the second column strength.

2. The translumenal access devices of claim 1, wherein the first column strength is sufficient to penetrate tissue.

3. The translumenal access device of claim 1, wherein the second column strength allows the second portion to buckle to prevent the hollow needle from further penetrating tissue.

4. The translumenal access device of claim 1, comprising a guide wire slidably disposed within the hollow needle, wherein the guide wire reinforces the column strength of the second portion to allow the second portion to further penetrate the tissue.

5. The translumenal access device of claim 1, wherein the cannula comprises an inflatable member at the distal end of the cannula.

6. The translumenal access device of claim 1, wherein the second portion of the hollow needle comprises at least one slit in the second portion to reduce the column strength of the second portion.

7. The translumenal access device of claim 6, wherein the at least one slit comprises a helical slit.

8. The translumenal access device of claim 6, wherein the at least one slit comprises a plurality of material removing slits.

9. The translumenal access device of claim 1, wherein the second portion of the hollow needle comprises a biasing member disposed in the second portion to reduce the column strength of the second portion.

10. The translumenal access device of claim 1, wherein the hollow needle comprises a third portion including a flexible portion with a third column strength disposed proximal to the second portion, the third column strength is greater than the second column strength.

11. The translumenal access device of claim 10, wherein the third portion of the hollow needle comprises a second helical slit in the third portion to reduce the column strength of the third portion.

12. The translumenal access device of claim 1, wherein the hollow needle is formed from a superelastic material and has a pre-curved shape.

13. A surgical instrument having proximal and distal ends defining an axis therebetween, wherein the surgical instrument is flexible and sized for insertion into a working channel of a flexible endoscope, comprising:
a cannula having a first channel extending from a proximal end of the cannula to a distal end of the cannula, wherein the first channel is adapted to retain a hollow needle; and
the hollow needle, slidably disposed within the cannula, comprising: a first portion including a sharpened rigid distal portion with a first column strength, a second portion including a floppy portion with a second column strength, the second portion disposed proximal to the first portion, wherein the first column strength is greater than the second column strength, and a second channel extending from a proximal end of the hollow needle to a distal end of the hollow needle, wherein the second channel is adapted to slidably retain a guide wire.

14. The surgical instrument of claim 13, wherein the first column strength is sufficient to penetrate tissue, and the second column strength allows the second portion to buckle to prevent the hollow needle from further penetrating tissue.

15. The surgical instrument of claim 13, wherein the guide wire reinforces the column strength of the second portion to allow the second portion to further penetrate the tissue.

16. The surgical instrument of claim 13, wherein the second portion of the hollow needle comprises at least one slit in the second portion to reduce the column strength of the second portion.

17. The surgical instrument of claim 13, wherein the second portion of the hollow needle comprises a biasing member disposed in the second portion to reduce the column strength of the second portion.

18. The surgical instrument of claim 13 wherein the hollow needle is formed from a superelastic material and has a pre-curved shape.

19. A method comprising:
inserting an endoscope into a lumen of a patient;
inserting a surgical instrument into the lumen of the patient through a working channel of the endoscope;
placing a cannula near a portion of tissue to be penetrated;
pressing the surgical instrument against the tissue;
advancing an endoscopic needle distally from the cannula;
penetrating the tissue with a first rigid portion of the needle;
further advancing the endoscopic needle distally to expose a second floppy portion of the needle;
allowing the second floppy portion to buckle;
inserting the surgical instrument through the penetration in the tissue until an inflatable member extends from one side of the penetration to another side of the penetration;
inflating the inflatable member;
placing a distal end of the endoscope at a proximal end of the inflatable member;
forcing the inflatable member and the distal end of the endoscope through the penetration;
deflating the inflatable member; and
removing the surgical instrument from the working channel of the endoscope.

20. The method of claim 18, further comprising:
sterilizing the surgical instrument; and
storing the surgical instrument in a sterile container.
